(19) United States
(12) Patent Application Publication
(10) Pub. No.: US 2015/0150501 A1
(43) Pub. Date: Jun. 4, 2015

(54) SLEEP DISORDER APPLIANCE COMPLIANCE

(71) Applicant: UNITED SCIENCES, LLC, ATLANTA, GA (US)

(72) Inventors: EOHAN GEORGE, ATLANTA, GA (US); KAROL HATZILIAS, ATLANTA, GA (US); MAYOOR PATEL, ATLANTA, GA (US); GOVINDA PINGALI, ATLANTA, GA (US); BRIAN POZGAY, ATLANTA, GA (US); JIM RAUBOLT, ATLANTA, GA (US); WESS ERIC SHARPE, VININGS, GA (US); JACOB THOMPSON, ATLANTA, GA (US)

(73) Assignee: UNITED SCIENCES, LLC, ATLANTA, GA (US)

(21) Appl. No.: 14/094,269

(22) Filed: Dec. 2, 2013

(51) Int. Cl. A61B 5/00

(52) U.S. Cl. CPC A61B 5/4833; A61B 5/4818

(57) ABSTRACT

Methods and apparatus are described for determining sleep disorder appliance compliance. Embodiments include receiving, in a sleep disorder compliance module through one or more sensors of an earpiece worn within an ear of a sleeping patient, information regarding the sleep of the patient; deriving, by the sleep disorder compliance module from the information regarding the sleep of the patient, one or more biometric values capable of indicating whether the patient is using a sleep disorder appliance; and determining, by the sleep disorder compliance module, whether the one or more biometric values indicate that the sleeping patient is presently using the sleep disorder appliance.

### BACKGROUND

In medicine, compliance describes the degree to which a patient correctly follows medical advice. Most commonly, it refers to medication or drug compliance, but it can also apply to other situations such as medical device use, self care, self-directed exercises, or therapy sessions. Worldwide, non-compliance is a major obstacle to the effective delivery of health care. Estimates from the World Health Organization (2003) indicate that only about 50% of patients with chronic diseases living in developed countries follow treatment recommendations.

### SUMMARY

Methods and apparatus are described for determining sleep disorder appliance compliance. Embodiments include receiving, in a sleep disorder compliance module through one or more sensors of an earpiece worn within an ear of a sleeping patient, information regarding the sleep of the patient; deriving, by the sleep disorder compliance module from the information regarding the sleep of the patient, one or more biometric values capable of indicating whether the patient is using a sleep disorder appliance; and determining, by the sleep disorder compliance module, whether the one or more biometric values indicate that the sleeping patient is presently using the sleep disorder appliance.

The foregoing and other objects, features, and advantages of the invention will be apparent from the following more detailed descriptions of example embodiments as illustrated in the accompanying drawings. In the drawings, like reference numbers generally represent like parts of example embodiments.

### BRIEF DESCRIPTION OF THE DRAWINGS

FIG. 1 sets forth a line drawing of example apparatus for determining sleep disorder appliance compliance.

FIGS. 2 and 3 set forth flow charts illustrating example methods of determining sleep disorder appliance compliance.

### DETAILED DESCRIPTION OF EXAMPLE EMBODIMENTS

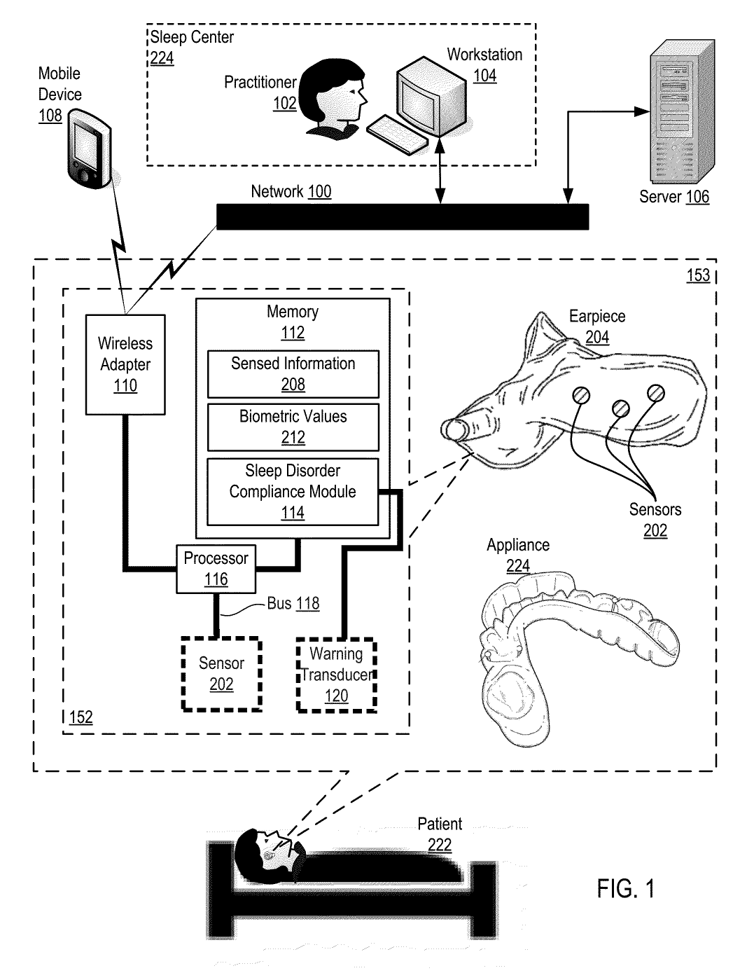

Example methods and apparatus or systems for determining sleep disorder appliance compliance are described with reference to the accompanying drawings, beginning with FIG. 1. FIG. 1 sets forth a line drawing of example apparatus for determining sleep disorder appliance compliance. The example apparatus of FIG. 1 includes an earpiece (204) that has sensors (202) integrated into the earpiece. The sensors are capable of sensing, when the earpiece is worn within an ear of a sleeping patient (222), information (208) regarding the sleep of the patient.

The earpiece (204) in this example is manufactured from a 3D image derived from an optical scan of the interior of the patient's ear canal. Creating a 3D image derived from an optical scan of the interior of the patient's ear canal can be carried out using methods and systems described in U.S. patent application Ser. Nos. 13/417,649; 13/417,767, 13/586,471; 13/586,411; 13/586,459; 13/546,448; 13/586,448; 13/586,474; 14/040,973, 14/041,943; 14/049,666; 14/049,530; 14/049,687, all incorporated by reference herein in their entirety.

The example apparatus of FIG. 1 also includes a sleep disorder compliance module (114) operably coupled to the sensors (202) through data bus (118). The sleep disorder compliance module is configured to derive from the sensed information (208) one or more biometric values (212). The biometric values are capable of indicating whether the patient (222) is using a sleep disorder appliance (224). The sleep disorder compliance module (114) is also configured to determine whether the biometric values indicate that the sleeping patient is presently using the sleep disorder appliance (224). The sleep disorder appliance (224) can be implemented in embodiments as, for example, an oral splint or a mandibular advancement splint to be worn in the patient's mouth during sleep.

In the example of FIG. 1, the sensed information (208) can include electroencephalography, electromyography, electrooculography, electrocardiography, accelerometry, reflective pulse oximetry, audio, temperature, and other sensed information as may occur to those of skill in the art. Also in the example of FIG. 1, the biometric values (212) can include pulse rate, body temperature, blood oxygen level, rapid eye movement sleep, non-rapid eye movement sleep, snoring, blood pressure, muscle tension, and other values derived from sensed information as may occur to those of skill in the art. In this example, the sleep disorder compliance module (114) is also configured to transmit, through a wireless data communications adapter (110) and a data communications network (100), the sensed information (208) as well as the biometric values (212) to a sleep center (224).

Examples of sleep disorders addressed by sleep disorder appliance compliance according to the example of FIG. 1 include sleep hypopnea, sleep apnea, and a sleep disorder that is a precursor to an episode of sleep apnea. This paper tends to focus on apnea and hypopnea, but there are many more sleep disorders and related disorders amenable to appliance compliance, including, for example, epileptiform discharges, seizures, RBD, REM without atonia, multiple parasomnias (sleep terrors, sleep walking, sleep talking, catathrenia, exploding head syndrome, confusional arousals, hypnogogic hallucinations, hypnopompic hallucinations, sleep paralysis, etc. . . . ), nocturnal movement disorders (bruxism, RLS, PLMD, muscle cramps, myoclonus, etc. . . . ), multiple causes for sleep fragmentation (pain-related insomnia, excessive cortical and sub-cortical arousal, hyperhidrosis, etc. . . . ), sleep-disordered breathing (all types including pediatric and adult), narcolpesy, cataplexy, delayed sleep phase, advanced sleep phase, idiopathic hypersomnolence, recurrent hypersomnolence, and day/night PSG testing also provides a host of other measures that are important such as EKG arrhythmias, peripheral O2/CO2 levels, respiratory drive via direct measures (RIP bands, intercostal EMG) and indirect measures (nasal airflow, air temperature flow, snoring), and periodic muscle analysis with EMG.

The sleep disorder compliance module (114) in the example of FIG. 1 is also configured to prevent an episode of sleep apnea through a warning implemented through a warning transducer (120) to the patient before an onset of sleep apnea when the sleep disorder compliance module determines that a precursor condition is present. Examples of warning transducers include a tone generator and speaker or earphone, a vibrator, a buzzer, or the like, integrated within the earpiece.

In the example of FIG. 1, the sleep disorder compliance module is disposed within an earpiece that is mounted in the patient's ear. In some embodiments, however, the sleep administration module is mounted in a mobile device (108), such as a smartphone, for example, and disposed within the patient's own residence, hotel room, or anywhere the patient may sleep. In such embodiments, the patient can administer sleep disorder appliance compliance anywhere, at home, traveling, and so on.

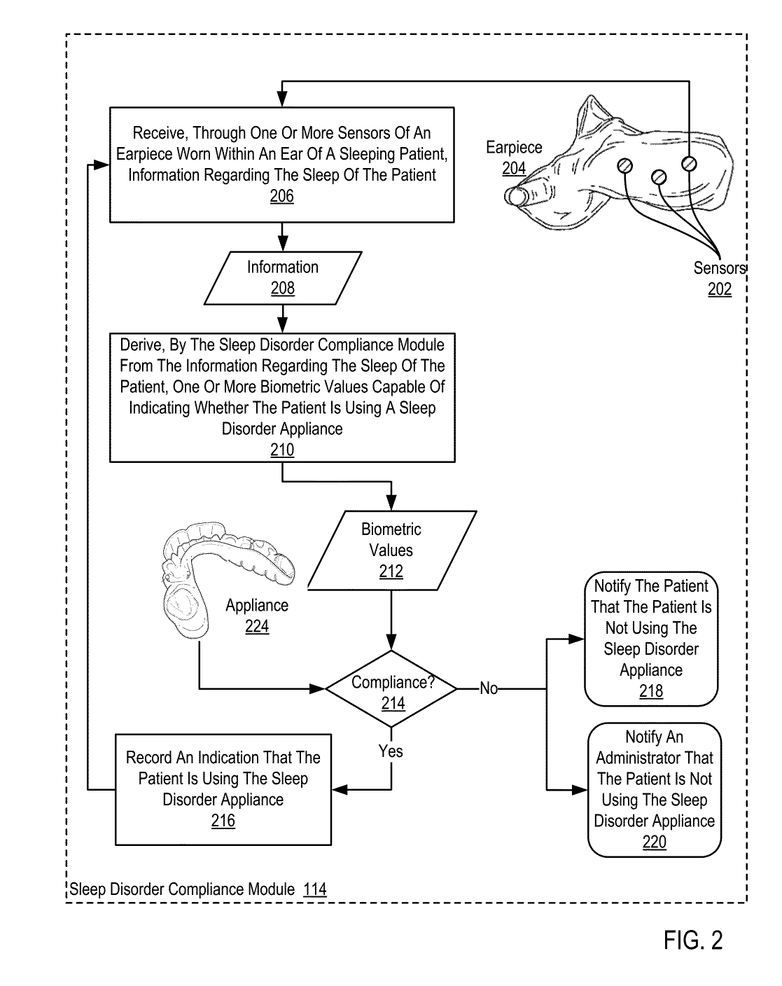

For further explanation, FIG. 2 sets forth a flow chart illustrating an example method of determining sleep disorder appliance compliance. The method of FIG. 2 includes receiving (206), in a sleep disorder compliance module (114) through one or more sensors (202) of an earpiece (204) worn within an ear of a sleep disorder patient, information (208) regarding the sleep of the patient.

The example method of FIG. 2 also includes deriving (210), by the sleep disorder compliance module (114) from the information (208) regarding the sleep of the patient (222), one or more biometric values (212) capable of indicating whether the patient is using a sleep disorder appliance. In the example of FIG. 2, the sensed information (208) can include electromyography, audio, piezoelectricity, and other sensed information as may occur to those of skill in the art. In the example of FIG. 2, the biometric values (212) can include jaw muscle tension, snoring, ear canal shape, and other values derived from sensed information as may occur to those of skill in the art.

The example of FIG. 2 includes determining (214), by the sleep disorder compliance module (114), whether the one or more biometric values (212) indicate that the sleeping patient (222) is presently using the sleep disorder appliance (224). Examples of sleep disorders amenable to the example of FIG. 2 include sleep hypopnea, sleep apnea, and a sleep disorder that is a precursor to an episode of sleep apnea. The method of FIG. 2 also includes recording (216), by the sleep disorder compliance module (114), an indication that the patient is using the sleep disorder appliance (224), such as, for example, making a record in computer memory, in a sleep administration database, or in a medical database. The method of FIG. 2 also includes notifying (218), by the sleep disorder compliance module (114), the patient that the patient is not using the sleep disorder appliance (224), such as, for example, by use of a warning transducer like the one illustrated at reference (120) on FIG. 1 and described above. The method of FIG. 2 also includes notifying (220), by the sleep disorder compliance module (114), an administrator that the patient is not using the sleep disorder appliance (224), that is, notifying a sleep disorder administrator or technologist, such as, for example, the patient's doctor, company, hospital, medical database in cyberspace, and so on.

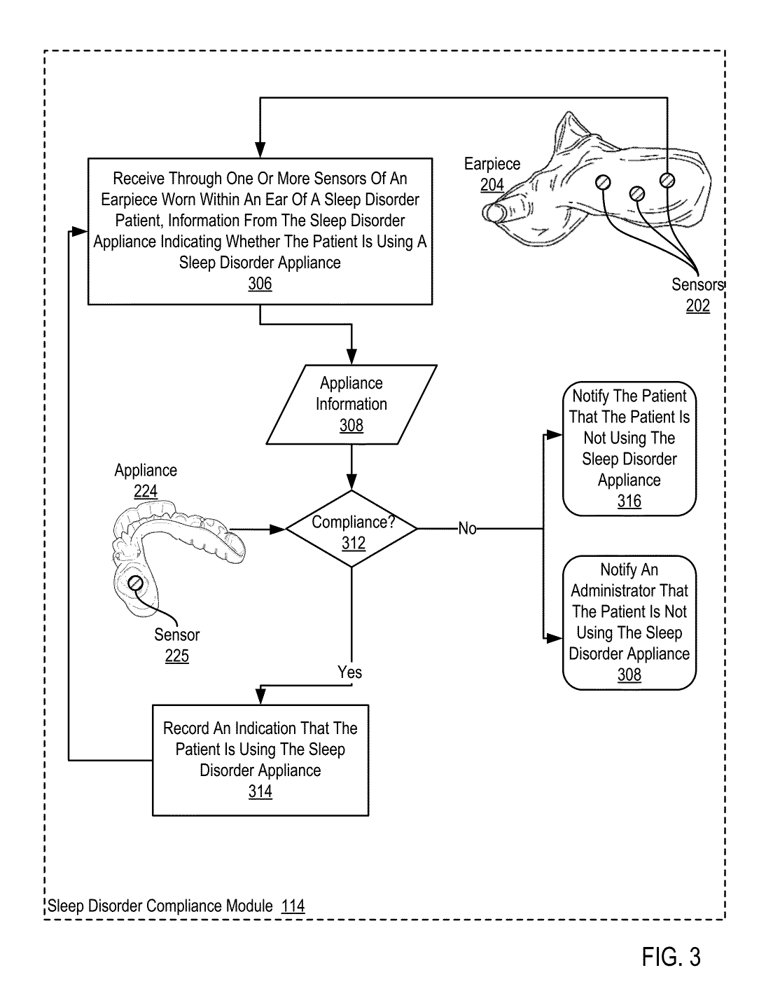

For further explanation, FIG. 3 sets forth a flow chart illustrating a further example method of determining sleep disorder appliance compliance. The method of FIG. 3 includes receiving (306), in a sleep disorder compliance module (114) through one or more sensors (202) of an earpiece (204) worn within an ear of a sleep disorder patient, information (308) from the sleep disorder appliance (224) indicating whether the patient is using a sleep disorder appliance (224).

The example of FIG. 3 includes determining (312), by the sleep disorder compliance module (114) based upon the received information (308), whether the patient is presently using the sleep disorder appliance (224). The information (308) can be provided from a switch, a pressure sensor, or other sensor (225) installed in the appliance (224) so that the switch or sensor is activated when the appliance is placed in the patient's mouth in preparation for sleep.

The method of FIG. 3 also includes recording (314), by the sleep disorder compliance module (114), an indication that the patient is using the sleep disorder appliance (224), such as, for example, making a record in computer memory, in a sleep administration database, or in a medical database. The method of FIG. 3 also includes notifying (316), by the sleep disorder compliance module (114), the patient that the patient is not using the sleep disorder appliance (224), such as, for example, by use of a warning transducer like the one illustrated at reference (120) on FIG. 1 and described above. The method of FIG. 3 also includes notifying (308), by the sleep disorder compliance module (114), an administrator that the patient is not using the sleep disorder appliance (224), that is, notifying a sleep disorder administrator or technologist, such as, for example, the patient's doctor, company, hospital, medical database in cyberspace, and so on.

Appliance compliance determinations are carried out generally in embodiments by use of electroencephalography (‘EEG’), electromyography (‘EMG’) and electrooculography (‘EOG’) information from sensors on an earpiece within the ear. The stage of sleep typically is taken from EEG and EMG information, from measures of the power of signals at certain frequencies. Stage 2 sleep will give sudden, short high-voltage wave bursts occurring at 12-14 Hz. Stage 3 sleep will show theta (4-7 Hz) and delta waves (1-4 Hz) with skeletal muscles very relaxed. Stage 4 is “slow wave sleep” because of delta waves, with a body turn approximately every 20 minutes. Rapid eye movement (‘REM’) sleep is indicated after the first four stages when frequency goes back to alpha waves, body temperature increases, heart rate increases, respiratory rate increases, blood pressure increases, the brain uses even more oxygen than when awake, eyes move rapidly. This particular signal from eye movement may be classified as EMG rather than EOG and is easily detected with information from the earpiece sensors.

Regarding REM sleep, sleep alternates between REM and non-REM or NREM; REM occurs about every 90 minutes and increases in length from 5-10 minutes to 20-50 minutes. The amount of REM sleep typically is determined in embodiments from sensor information by detecting eye movement using EOG and EMG. A person feels most rested when awakened just after a REM cycle, so that warnings can signal a person to awaken when apparatus in embodiments detects that REM is finished.

Clenching and grinding of teeth is detected in embodiments by use of EMG. For each skeletal muscle, there is an optimal longitudinal length at which the maximum muscle activation can occur; muscle activation of the muscles of mastication can be measured using EMG. When placing a sleep disorder appliance into the mouth, the teeth become separated, slightly lengthening the muscles of mastication, preventing the electrical signal from the muscles of mastication from being as intense as having no teeth separation. For a patient that is prone to clenching, the clenching intensity will be decreased when wearing the oral appliance. Warnings to the patient in embodiments effectively implements relaxation training. Some embodiments play music or tones only when a patient is relaxed (or vice versa) using EMG detection of nearby muscle activity (muscles of mastication).

Accelerometry from within the ear includes in embodiments nine degrees of freedom (9DOF accelerometry). 9DOF accelerometry includes multiple axes of detection from which, based on acceleration due to gravity, a patient's resting head position can be determined. Then embodiments can alert the patient to changes into nonoptimal sleep positions.

Oximetry typically is implemented as reflection pulse oximetry from within the ear or transmission pulse oximetry around the pinna. Embodiments can use both red (600-750 nm) and infrared light (850-1000 nm) to illuminate blood and use a photosensor to measure either transmission or reflection. Red light at 660 nm reflects off of hemoglobin when it is saturated (HbO2) and infrared light at 940 nm reflects off of de-oxygenated hemoglobin (Hb).

$$Ratio of Ratios ∼ \frac{ln ( \frac{{Red}_{systole}}{{Red}_{diastole}} )}{ln ( \frac{{IR}_{systole}}{{IR}_{diastole}} )} \qquad Formula 1$$

The ‘ratio of ratios’ according to Formula 1 is calibrated in embodiments to determine peripheral capillary oxygen saturation or SpO₂ in percentage, using a lookup table to determine the actual percentage. SpO₂ (%) can be measured, a value that decreases during an apneic episode. Pulse rate (beats per minute) can be measured in embodiments with oximetry because there is variable light absorption due to pulsatile volume of arterial blood. When measuring from within the ear canal, direct reflective pulse oximetry towards the superficial temporal artery, which runs anterior to the canal, or associated vasculature. When measuring in locations requiring light transmission detection (instead of reflection), such as through the pinna or ear lobe, embodiments use a clip that places lights on one side of tissue and photosensor on the other side. While using an oral appliance for obstructive sleep apnea, there are no acute decreases in oxygen saturation unless sleep apnea occurs via central sleep apnea where there is no respiratory effort by the patient. Embodiments therefore can alert a patient when oxygen saturation decreases below a threshold.

Sensors in embodiments can include a microphone to sense or record snoring sounds. Snoring sounds decrease with use of an obstructive sleep apnea oral appliance. Snoring sounds can also be used to indicate oral appliance (mandibular advancement appliance) effectiveness at maintaining pharyngeal patency. Audio from snoring in embodiments can complement accelerometer information to determine patient movements during sleep, alerting a patient to change positions when snoring indicates nonoptimal body position.

Additional warning-type technology in embodiments can include a speaker or earphone integrated in the earpiece that delivers information directly into a patient's ear without disrupting others nearby. Audible warnings can include alerts to change sleeping position, alerts to wake a patient, music or relaxation sounds, including playing slow breathing sounds for breath matching, to aid a patient in falling asleep. These alerts and sounds in embodiments are implemented with a phone paired via Bluetooth with a source of soothing sounds or music and, in some embodiments, are supportive of sleep-related training such as EMG relaxation training.

An embodiment includes a Piezo sensor to detect pulse from within the ear. This is in addition to pulse oximetry which in some embodiments may have too low measurement/calculation frequency or too low noise for pulse detection. A Piezo sensor is mounted on the earpiece so as to contact skin in the ear canal and detect pulse through impulses affecting skin pressure on the sensor. In at least one embodiment, skin pressure noise from snoring, movement, and the like, is canceled with audio noise from a microphone.

In some embodiments, earpiece sensors can include one or more active in-ear readers for sensors mounted on an oral appliance and directed to sleep disorder appliance compliance, including a passive RadioFrequency Identification (RFID) tag, a Near Field Communications (‘NFC’) tag, a contactless smart card, or the like, attached to the oral appliance and registered with the active in-ear reader in an earpiece when in use to determine appliance compliance. A passive tag in an embodiment is switched on only when the oral appliance is locked into the patient's mouth, working only when two pieces of the RFID tag are connected to each other via electrodes to the gums. One part of such an RFID tag is attached to the patient, making electrical contact to a second part of the RFID tag mounted on the oral appliance only when the appliance is worn. In another embodiment, a passive RFID tag is split into two parts as an open circuit, and the act of placing the oral appliance in the mouth and pressing it onto the teeth mechanically connects the two for further operation with an active RFID reader.

The active in-ear reader in the earpiece sends an RF signal to power a passive RFID or NFC tag installed on the oral appliance. The active in-ear reader can send an RF signal that powers a passive tag on the oral appliance, with the passive tag connected to one or more physiological sensors, temperature, O2, pressure against teeth, electrical conduction, and so on, with the sensor data then sent back to the active reader in the ear. A force sensor may be embedded in an oral appliance to be pressed against tooth during use, with force data be transferred to the in-ear reader to determine appliance compliance. A temperature sensor may be embedded into an oral appliance, with temperature data transferred to the in-ear reader to determine appliance compliance.

In some embodiments, an oral appliance contains a piezo or bone conduction transducer, with audio vibrations received by microphone in the ear or on the appliance, with a connection to the earpiece by RFID, ultrasound, vibration, and so on. An ultrasound signal in such embodiments is sent from the ear device through the body and makes contact with the oral appliance. The signal is then passively modulated and reflected through the body and back to the ear device. The modified signal received by the ear device confirms proper placement of the oral appliance in the mouth.

Because a specific ear canal geometry exists with each jaw position, and an appliance such as a mandibular advancement oral appliance significantly changes jaw position, embodiments can determine whether a patient is wearing an oral appliance by analyzing the shape of the ear canal. Apparatus for appliance compliance monitoring can be configured to scan the ear while the patient wears the oral appliance, with several force sensors placed around the shell of the appliance. The force values associated with placement of the oral appliance are calibrated per patient and per appliance. In some embodiments the force sensors are capacitive touch sensors rather than force sensors as such, although persons of skill in the art may think of other kinds of sensors. Such embodiments also encourage use of the ear device while wearing the oral appliance because the best fit will be obtained in the ear canal when wearing the oral appliance. Jaw position associated with not wearing the oral appliance would throw off contact of at least one of the EEG/EMG/EOG electrodes with the skin—if so, this would be determined from the sensed information and/or biometric values.

Also in some embodiments, appliance compliance is measured by aiming ultrasound toward the mandibular condyle from within the ear, from a transducer in the earpiece. The distance to the condyle corresponds with use of the oral appliance and is calibrated per patient and per appliance.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

What is claimed is:

1. A method of determining sleep disorder appliance compliance, the method comprising:
receiving, in a sleep disorder compliance module through one or more sensors of an earpiece worn within an ear of a sleeping patient, information regarding the sleep of the patient;
deriving, by the sleep disorder compliance module from the information regarding the sleep of the patient, one or more biometric values capable of indicating whether the patient is using a sleep disorder appliance;
determining, by the sleep disorder compliance module, whether the one or more biometric values indicate that the sleeping patient is presently using the sleep disorder appliance.

2. The method of claim 1 wherein the information comprises electromyography, audio, and piezoelectricity.

3. The method of claim 1 wherein the biometric values comprise jaw muscle tension, snoring, and ear canal shape.

4. The method of claim 1 wherein the sleep disorder is sleep apnea.

5. The method of claim 1 wherein the sleep disorder is sleep hypopnea.

6. The method of claim 1 wherein the sleep disorder appliance is an oral splint.

7. The method of claim 1 further comprising recording, by the sleep disorder compliance module, an indication that the patient is using the sleep disorder appliance.

8. The method of claim 1 further comprising notifying, by the sleep disorder compliance module, the patient that the patient is not using the sleep disorder appliance.

9. The method of claim 1 further comprising notifying, by the sleep disorder compliance module, an administrator that the patient is not using the sleep disorder appliance.

10. A method of determining sleep disorder appliance compliance, the method comprising:
receiving, in a sleep disorder compliance module through one or more sensors of an earpiece worn within an ear of a sleep disorder patient, information from the sleep disorder appliance indicating whether the patient is using a sleep disorder appliance; and
determining, by the sleep disorder compliance module in dependence upon the received information, whether the patient is presently using the sleep disorder appliance.

11. The method of claim 10 wherein the information comprises radio frequency information.

12. The method of claim 10 wherein the sleep disorder is sleep apnea.

13. The method of claim 10 wherein the sleep disorder is sleep hypopnea.

14. The method of claim 10 wherein the sleep disorder appliance is an oral splint.

15. The method of claim 10 further comprising recording, by the sleep disorder compliance module, an indication that the patient is using the sleep disorder appliance.

16. The method of claim 10 further comprising notifying, by the sleep disorder compliance module, the patient that the patient is not using the sleep disorder appliance.

17. The method of claim 10 further comprising notifying, by the sleep disorder compliance module, an administrator that the patient is not using the sleep disorder appliance.

18. Apparatus for determining sleep disorder appliance compliance, the apparatus comprising:
an earpiece (204) having integrated sensors (202) capable of sensing, when the earpiece is worn within an ear of a sleeping patient (222), information regarding the sleep of the patient; and
a sleep disorder compliance module (114) operably coupled to the sensors (202) and configured to derive from the sensed information one or more biometric values capable of indicating whether the patient is using a sleep disorder appliance and determine whether the one or more biometric values indicate that the sleeping patient is presently using the sleep disorder appliance.

19. The apparatus of claim 18 wherein the sleep disorder is sleep apnea.

20. The apparatus of claim 18 wherein the sleep disorder appliance is an oral splint.
